(19) United States
(12) Patent Application Publication
(10) Pub. No.: US 2010/0120146 A1
(43) Pub. Date: May 13, 2010

(54) ACTIVATOR OF SIGNAL TRANSDUCTION PATHWAY

(75) Inventors: Hideyuki Okano, Tokyo (JP); Kazunobu Sawamoto, Tokyo (JP); Masanori Sakaguchi, Tokyo (JP); Jun Hirabayashi, Ibaraki (JP)

Correspondence Address: CLARK & ELBING LLP, 101 FEDERAL STREET, BOSTON, MA, 02110, US

(73) Assignee: KEIO UNIVERSITY, Minato-ku (JP)

(21) Appl. No.: 12/295,814

(22) PCT Filed: Apr. 4, 2007
(86) PCT No.: PCT/JP2007/057588

§ 371 Date: Jan. 22, 2010

(30) Foreign Application Priority Data
Apr. 5, 2006 (JP) 2006-104610

(51) Int. Cl. C12N 5/02

(52) U.S. Cl. 435/375

(57) ABSTRACT

The present invention is directed to providing the activator of the integrin signaling in neural stem cells or neural progenitors and the method for using the same. The activator of the integrin signaling comprises galectin-1 and can be used as a regulator that regulates maintenance, survival, or differentiation of a neural stem cells or a neural progenitor.

### CROSS REFERENCE TO RELATED APPLICATIONS

This application claims the benefit of priority to Japanese Patent Application No. 2006-104610, filed on Apr. 5, 2006, which is incorporated herein by reference.

### TECHNICAL FIELDS

The present invention relates to integrin β1 signaling activators.

### BACKGROUND ART

Regeneration of the injured central nervous system is difficult, but it has been reported in animal experiments that transplantation of embryo tissues, especially of neural stem cells, is effective. However, to obtain neural stem cells sufficient for therapy, many donations of aborted embryos are required. Moreover, since use of embryos causes an ethical issue, practical clinical application of neural stem cells is difficult.

Thus, as a candidate of a transplantation material replacing neural stem cells directly isolated from embryos, neural stem cells that have been cultured and have proliferated in vitro have been a focus of attention. Neural stem cells are undifferentiated cells with self-replication ability and pluripotency. Since they proliferate unlimitedly by in vitro culture, they enable supply of a sufficient number of donor cells.

In maintenance, survival, proliferation, and differentiation of neural stem cells or neural progenitors, integrin β1 plays an important role (Campos L. S. “Beta1 integrins and neural stem cells: making sense of the extracellular environment.” Bioessays vol. 27 No. 7 p. 698-707, 2005; Jacques T. S. et al. “Neural precursor cell chain migration and division are regulated through different betal integrins.” Development vol. 125 No. 16 p. 3167-3177, 1998; Gimond C. et al.

“Defects in adhesion and migration, but not in proliferation and differentiation, of embryonic stem cells upon replacement of integrin subunit beta1A by beta1D.” Differentiation. Vol. 66 No. 2-3 p. 93-105, 2000; Prestoz L. et al. “Association between integrin-dependent migration capacity of neural stem cells in vitro and anatomical repair following transplantation.” Mol. Cell Neurosci. Vol. 18 No. 5 p. 473-484, 2001; Murase S. and Horwitz A. F. “Deleted in colorectal carcinoma and differentially expressed integrins mediate the directional migration of neural precursors in the rostral migratory stream.” J. Neurosci. Vol. 22 No. 9 p. 3568-3579, 2002; Yoshida N, et al. “Decrease in expression of alpha 5 beta 1 integrin during neuronal differentiation of cortical progenitor cells.” Exp. Cell Res. Vol. 287 No. 2 p. 262-271, 2003; Campos L. S. et al. “Beta1 integrins activate a MAPK signalling pathway in neural stem cells that contributes to their maintenance.” Development. Vol. 131 No. 14 p. 3433-3444, 2004; Yanagisawa M. et al. “Roles of lipid rafts in integrin-dependent adhesion and gp130 signalling pathway in mouse embryonic neural precursor cells.” Genes Cells. Vol. 9 No. 9 p. 801-809, 2004; Tate M. C. et al. “Specific betal integrins mediate adhesion, migration, and differentiation of neural progenitors derived from the embryonic striatum.” Mol. Cell Neurosci. Vol. 27 No. 1 p. 22-31, 2004; Andressen C. et al. “The contribution of betal integrins to neuronal migration and differentiation depends on extracellular matrix molecules.” Eur. J. Cell Biol. Vol. 84 No. 12 p. 973-982, 2005; Campos L. S. et al. “Notch, EGFR and beta 1 integrin pathways are coordinated in neural stem cells.” J. Biol. Chem. accepted manuscript version (Dec. 6, 2005). Thus, it is inferred that, by regulating integrin β1 signaling, regulation of maintenance, survival, proliferation, or differentiation of neural stem cells or neural progenitors should become possible.

### DISCLOSURE OF THE INVENTION

### Problems to be Solved by the Invention

Thus, the object of the present invention is to provide integrin β1 signaling activators in a neural stem cell or a neural progenitor as well as regulators which regulate maintenance, survival, or differentiation of a neural stem cell or a neural progenitor through integrin β1 signaling.

### Means for Solving the Probles

The inventors have already found that galectin-1 is one of the factors responsible for neural stem cell proliferation. Thus, as a result of diligent study to attempt to identify the receptor for galectin-1, they have revealed that the galectin-1 receptor is integrin β1. The present invention has thus been accomplished.

The activator according to the present invention activates integrin β1 signaling in a neural stem cell or a neural progenitor, and contains galectin-1. In the activation method according to the present invention, which activates integrin β1 signaling in a neural stem cell or a neural progenitor in culture, galectin-1 is administered to the cell.

Further, the regulator according to the present invention regulates maintenance, survival, or differentiation of a neural stem cell or a neural progenitor, and contains galectin-1. In the regulating method according to the present invention, which regulates maintenance, survival, or differentiation of a neural stem cell or a neural progenitor, galectin-1 is administered to the cell.

### BRIEF DESCRIPTION OF THE DRAWINGS

FIG. 1 shows examples of experiments demonstrating that integrin 1 is a receptor for galectin-1: (A) an example of experiment showing that the binding of galectin-1 to neural stem cells is inhibited by lactose in vivo; (B) an example of experiment showing that integrin β1 is bound to a CS-GAL-1 affinity column and then eluted; and (C) an example of experiment showing that cells expressing integrin-1 have affinity to galectin-1.

FIG. 2 shows a result obtained by allowing BrdU to be incorporated in neural stem cells at the same time when galectin-1 was infused into a brain in the Example: (A) shows the method for administering BrdU and galectin-1 in the Example; (B) shows a result of in situ visualization of proliferating cells; and (C) shows a result of counting the number of BrdU-positive nuclei.

FIG. 3 shows a result obtained by allowing BrdU to be incorporated into a brain at the same time when galectin-1 and anti-integrin antibody were infused into the brain in the Example: (A) shows a result of in situ visualization of proliferating cell; and (B) shows a result of counting the number of BrdU-positive nuclei.

### BEST MODE OF CARRYING OUT THE INVENTION

Integrins are receptor proteins composed of a subunit and β subunit heterodimers. Among the integrin subunits, integrin β1 has been shown to play a crucial role in the developmental process.

### Regulator Which Regulates Maintenance Of Neural Stem Cells Or Neural Progenitors

In the developing cortex, neural stem cells in the ventral zone (VZ) region first divide symmetrically, and, next, divide asymmetrically to give rise to a neural stem cell and a neural progenitor, which maintain neural stem cells. Integrin-linked kinase (ILK), an intracellular protein interacting with β1, is known to suppress transcriptional expressions of E-cadherin and β catenin by upregulating expressions of transcriptional repressors Snail/Slug. This mechanism is suggested to underlie the phenomenon that VZ cells with high expression levels of E cadherin/β catenin become able to migrate (Campos L. S., Bioessays 27, 698-707, 2005) . Meanwhile, it is known that overexpression of β catenin in the presence of FGF2 keeps neural progenitors proliferating, while overexpression of β catenin in the absence of FGF2 steers them toward neuronal differentiation. Since administration of FGF to neural progenitors upregulates the integrin β1 expression, the involvement of FGF in the effect by β catenin can be partly attributed to the cell-cell or cell-ECM microenvironment. Taken together, these findings suggest that a network of protein interactions functions in the VZ and that integrin β1 signaling chooses whether to maintain stem cells or to help them differentiate/migrate through cross-talk with other signaling pathways (Campos L. S., Bioessays 27, 698-707, 2005).

Therefore, by activating integrin β1 by galectin-1, the state of neural stem cells or neural progenitors can be maintained; thus galectin-1 is effective as a regulator that regulates maintenance of neural stem cells or neural progenitors.

### Regulator That Regulates Survival Of Neural Stem Cells Or Neural Progenitors

Symmetric and asymmetric divisions of neural stem cells in the VZ region can be regulated by regulating cell survival by asymmetrically localizing specific molecules. It is known that during the developmental process a basal lamina regulates programmed cell death (PCD). Only cells remaining close to a basal lamina or those residing in an environment where other molecules (for example growth factors) are present will survive, and cell death is triggered in cells that have lost contact with the appropriate matrix. In fact, loss of integrin signaling can induce PCD (Zhang Z., Proc. Natl. Acad. Sci. USA 92, 6161-6165, 1995; Gilmore A. P. et al., J. Cell Biol. 149, 431-446, 2000; Gary D. S. et al., J. Neurochem 84, 878-890, 2003) and altered conformation of integrin β1 can cause apoptosis (Prince J. M. et al., Dev. Dyn. 223, 497-516, 2002). In addition, free integrin molecules recruit caspase-8 to the membrane and activates it, while integrin binding inhibits this integrin-caspase interaction, thereby increasing cell survival (Stupack D. G. et al., J. Cell Biol. 155, 459-470, 2004). Furthermore, it is known that α3β1 integrin increases cell survival by activating MAPK signaling (Manohar A. et al., J. Cell Sci. 117, 4043-4054, 2004). Taken together, these findings suggest that, in vivo as well, survival of neural stem cells or neural progenitors is maintained by integrin β1 in the VZ in which integrin β1 is highly expressed (Campos L. S., Bioessays 27, 698-707, 2005).

Therefore, by activating integrin β1 by galectin-1, survival of neural stem cells or neural progenitors can be kept; thus galectin-1 is effective as a regulator that regulates survival of neural stem cells or neural progenitors.

### Regulator That Regulates Differentiation Of Neural Stem Cells Or Neural Progenitors

It has recently been clarified that when the expression levels of integrin β1 are reduced in neural progenitors, the number of nestin-positive cells is decreased (Leone D. et al. J. Cell Sci. 118, 2005). Nestin is a marker for the undifferentiated state of a neural stem cell or a neural progenitor. Thus this experimental result demonstrates that integrin β1 is required for maintenance of the undifferentiated states of neural stem cells and neural progenitors.

Therefore, by activating integrin β1 by galectin-1, the undifferentiated state of neural stem cells or neural progenitors can be maintained; thus galectin-1 is effective as a regulator that regulates differentiation of neural stem cells or neural progenitors.

### EXAMPLES

### Example 1

### Integrin β1 Constitutes a Receptor of Galectin-1

To examine whether a sugar is involved in the binding of galectin-1 to neural stem cells, tissue staining was performed with biotinylated C2S galectin-1 (C2S-GAL-1-bio) in the presence of lactose (provided by the National Institute of Advanced Industrial Science and Technology). This C2S-GAL-1, a galectin-1 mutant in which cysteine at position 2 is replaced with serine (Hirabayashi & Kasai, J Biol. Chem. 266, 23648-23653, 1991), has been shown to have normal carbohydrate binding ability (Purkrabkova et al., Biol. Cell. 95, 535-545, 2003).

First, the brain was removed from 56-day to 105-day old mice, perfusion-fixed with 4% formaldehyde solution, post-fixed at 4° C. overnight in the same solution. Then 50 μm thick vibratome sections of the brain were prepared.

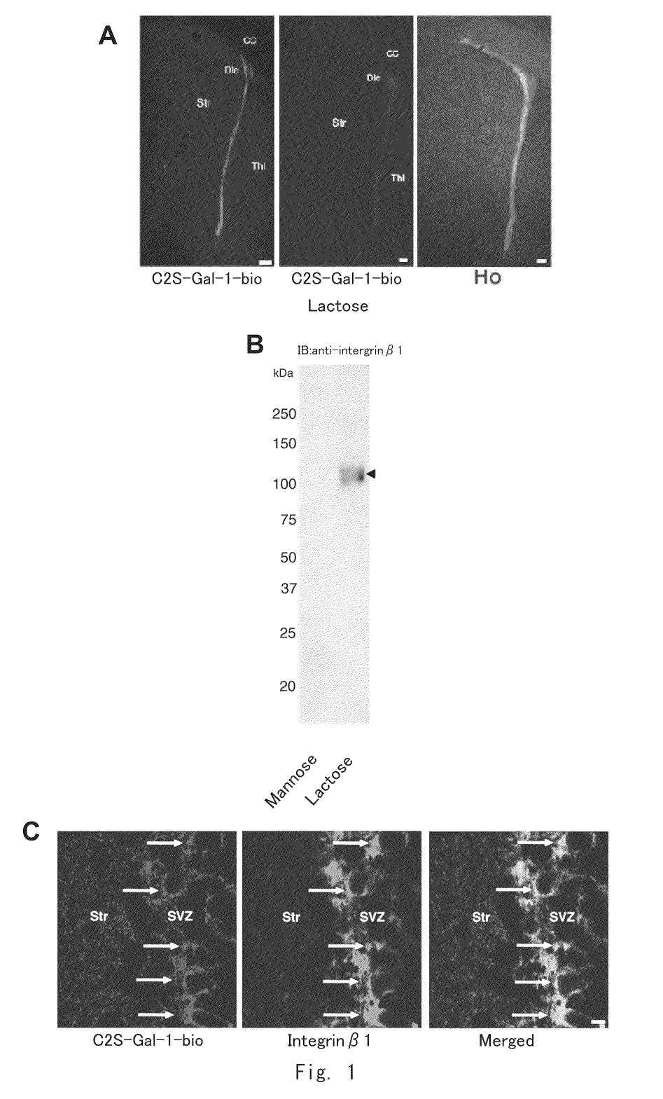

The sections were incubated at 4° C. overnight in a 1 μg/mL C2S-GAL-1-bio solution dissolved in PBS containing 0.5% Triton X-100 and 1% BSA in the presence of 20 mM lactose. Fluorescent staining was performed by using the Vectastain ABC kit (Vector Laboratories) and TSA (PerkinElmer, Inc.). As a result, the signals became significantly weak, as compared with the control that was treated with the solution without lactose (FIG. 1A). This indicates that the binding of galectin-1 to neural stem cells is inhibited by lactose.

To explore the mechanism of this inhibition of binding by lactose and to identify the galectin-1 binding factor, a galectin-1 binding factor was isolated from an extract of SVZ by using a C2S-GAL-1 affinity column. First, recombinant C2S-GAL-1 was immobilized on an NHS-activated Sepharose column (Amersham Biosciences), packed in a 2 ml volume column, and equilibrated with PBS containing 2 mM EDTA and 4 mM 2-mercaptoethanol (ME-PBS). Meanwhile, brains were removed from five adult mice, and SVZ tissues were excised with an ophthalmic scalpel under a stereoscopic microscope. After being sonicated, the SVZ tissues were washed three times by pipetting the tissues in ME-PBS containing 20 mM lactose (Wako), centrifuging them and removing the supernatant, and finally solubilized with ME-PBS containing 1% Triton X-100. The obtained extract was injected onto the column being equilibrated with ME-PBS. After nonspecifically bound molecules were removed by washing the column with ME-PBS containing 100 mM a-methylmannopyranoside (Sigma), the bound molecules were eluted with ME-PBS containing 20 mM lactose or 100 mM mannose (Wako). Western blotting was performed to examine whether integrin β1 was present in the effluent. Integrin β1 was detected by using anti-integrin antibody (mouse anti-integrin B1, IgG monoclonal, BD, 1:100 dilution) as the primary antibody) and HRP labeled anti-mouse IgG (Jackson Immunoresearch Labs, 1:500 dilution) as the secondary antibody. Whereas integrin β1 was eluted with lactose-containing ME-PBS, integrin β1 was not eluted with mannose-containing ME-PBS as shown in FIG. 1B. Considering that lactose inhibits the binding of galectin-1 to its receptor, at least integrin β1 was supposed to be a galectin-1 receptor on neural stem cells.

Next, the previously-described vibratome sections of mouse brain were doubly stained by using C2S-GAL-1 and the anti-integrin antibody (Pharmingen, 1:10 dilution). For the detection of C2S-GAL-1, the Vectastain ABC kit (Vector Laboratories) and TSA (PerkinElmer, Inc.) were used. For the detection of integrin, anti-mouse IgG antibody (Jackson, 1:500 dilution) was used as the secondary antibody. As shown in FIG. 1C, cells in which integrin β1 was expressed had affinity to C2S-GAL-1, supporting that the receptor for galectin-1 is integrin β1.

### Example 2

### Anti-Integrin β1 Antibody Inhibits the Interaction Between Galectin-1 And Integrin β1

It is known that, in the SVZ, a part of SVZ astrocytes function as neural stem cells; that is, they can differentiate into transit amplifying cells (TA cells) at an intermediate stage of differentiation, further proliferate, and differentiate into neuroblasts (NBs). These neural stem cells are known to proliferate comparatively slowly and thus can be identified by allowing them to incorporate BrdU for a long period.

Meanwhile, infusion of galectin into the mouse brain enhances proliferation of the neural stem cells in the SVZ (WO 2005/026343). Thus, the effect of inhibition of the interaction between galectin-1 and integrin β1 on proliferation of the neural stem cells was examined.

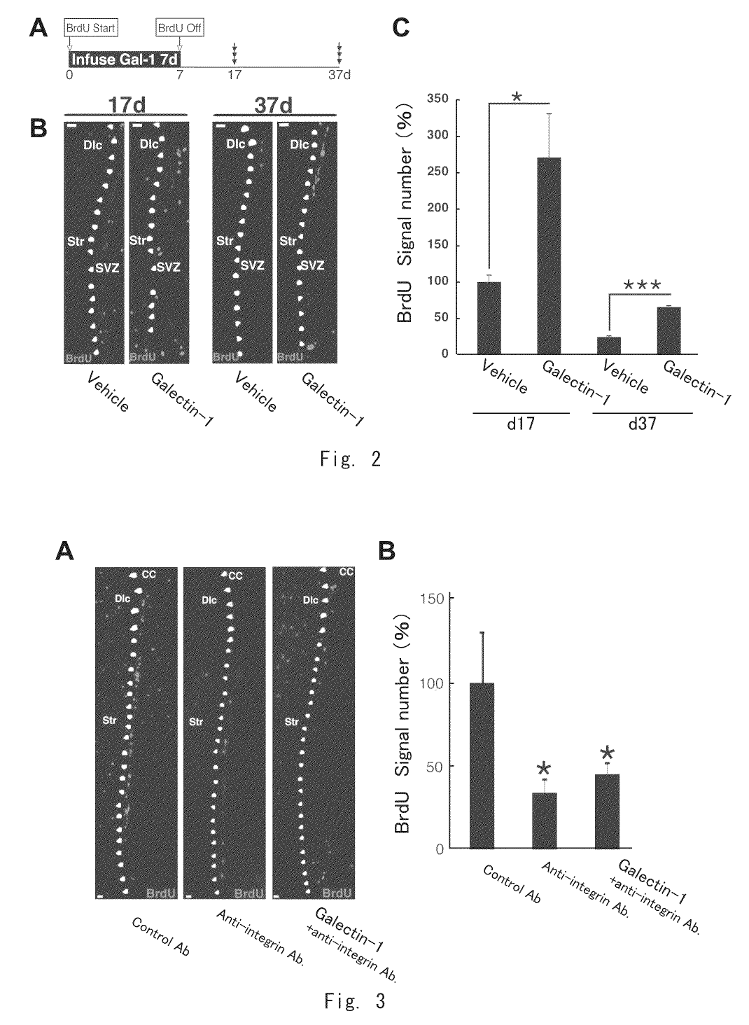

First, recombinant galectin-1 (2 or 14 μg), an anti-galectin neutralizing antibody (rabbit IgG, 30 μμg/ml, provided by Kirin Brewery), a control antibody which does not recognize galectin (rabbit IgG, 30 μg/ml, provided by Kirin Brewery), an anti-integrin β1 antibody (hamster IgM, 10 μg/ml, BD), and a control antibody which does not recognize integrin β1 (hamster IgM, 10 μg/ml, BD) were dissolved each in 0.9% saline containing 1 mg/ml mouse serum albumin (Sigma). Using stereotaxic surgery, a cannula was inserted and placed at a position 0.2 mm posterior and 0.8 mm lateral to the bregma and 2.0 mm deep from the skull surface, and the galectin solution was continuously infused into the lateral ventricle at a rate of 0.5 μl/h with an osmotic pump for 7 days (FIG. 2A).

To examine proliferation of neural stem cells in the above-described conditions, the proliferating cells were visualized by allowing BrdU to be incorporated in the cells for a long period. First, water containing 1 mg/ml BrdU was fed to mice as drinking water (FIG. 2A) throughout the 7-day continuous administration of galectin-1 (FIG. 2B and C), or throughout the 7-day continuous administration of galectin-1 plus anti-integrin antibody (FIG. 3A and B). Either 17 and 37 days (FIG. 2B and C) or 17 days (FIG. 3A and B) after the last day of the administrations, the mice were dissected and the brains were removed. Vibratome sections were prepared from the SVZ region as described above. Using a rat anti-BrdU monoclonal antibody (rat monoclonal antibody [BU 1/75 (ICR)], 1:100 dilution, Abcam, Inc.) as the primary antibody and a biotin-labeled anti-rat IgG antibody (1:100 dilution, Jackson ImmunoResearch Labs) as the secondary antibody, the sections were observed with a confocal laser microscope (LSM-510, Zeiss) (FIGS. 2B and 3A). In addition, to quantify signals, the numbers of BrdU-positive nuclei were counted on 1 μm thick cross sections taken at every 7 μm in the SVZ region (bregma+0 to +1) and the averages were plotted (FIGS. 2C and 3B).

As shown in FIG. 2B and C, in the mouse to which galectin was admministered, the number of slowly proliferating cells in the SVZ was significantly increased as compared with untreated control mice both on day 37 and on day 17(on day 17 p=0.01; on day 37 p<0.001). This result indicated that infusion of galectin-1 into the brain increases the number of neural stem cells.

Meanwhile, as shown in FIG. 3A and B, in the mice to which the anti-integrin antibody was administered, the number of slowly proliferating cells was significantly reduced in the SVZ, as compared with the untreated control mice (p<0.05). This result indicated that infusion of the anti-integrin antibody into the brain reduces the number of neural stem cells.

Moreover, when galectin-1 and the anti-integrin antibody were concomitantly administered, the effect of galectin-1 was not exerted and the number of slowly proliferating cells was significantly reduced (p<0.05). This result indicated that the decrease in the number of neural stem cells due to anti-integrin antibody was caused by the inhibition of the interaction between galectin-1 and integrin by the anti-integrin antibody.

### <Conclusion>

These findings revealed that in neural stem cells or neural progenitors, integrin β1 is a receptor for galectin-1 and that, galectin-1 activates integrin signaling by binding to integrin β1.

### INDUSTRIAL APPLICABILITY

The present invention has made it possible to provide integrin β1 signaling activators in neural stem cells or neural pregenitors as well as regulators that regulate maintenance, survival, or differentiation of neural stem cells or neural progenitors through integrin β1 signaling.

1. A method for activating integrin β1 signaling in a neural stem cell or a neural progenitor cell, comprising administering galectin-1 to the cell.

2. A method for regulating maintenance, survival, or differentiation of a neural stem cell or a neural progenitor cell, comprising administering galectin-1 to the cell.

3. (canceled)

4. (canceled)
